(19) United States
(12) Patent Application Publication
(10) Pub. No.: US 2006/0009827 A1
(43) Pub. Date: Jan. 12, 2006

(54) Method and apparatus for anchoring of pacing leads

(76) Inventors: Paul A. Kurth, Santa Barbara, CA (US); Seth J. Worley, Lancaster, PA (US)

Correspondence Address: MYERS DAWES ANDRAS & SHERMAN, LLP, 19900 MACARTHUR BLVD.,, SUITE 1150, IRVINE, CA, 92612, US

(21) Appl. No.: 10/497,763

(22) PCT Filed: Nov. 17, 2003
(86) PCT No.: PCT/US03/37045

§ 371 Date: Aug. 25, 2005

Related U.S. Application Data
(60) Provisional application No. 60/426,773, filed on Nov. 15, 2002.
(60) Provisional application No. 60/464,437, filed on Apr. 22, 2003.
(60) Provisional application No. 60/476,487, filed on Jun. 6, 2003.
(60) Provisional application No. 60/479,399, filed on Jun. 18, 2003.

(51) Int. Cl. A61N 1/05

(52) U.S. Cl. 607/119

(57) ABSTRACT

A pacemaker lead (18) is implanted by using a two anchor system. One anchor (916) to first secure the position of an introducer (14) through which the pacemaker lead will be implanted, and a second anchor (20) used for implanting the distal tip of the pacemaker lead (18). Examples of the anchors include various types of mechanical anchors or mechanical anchor combinations such as a suction anchor, a barbed needle, or a needle and balloon combination. The anchors can be used in the pericardial space in the heart by disposing an elongate instrument into the venous system of the heart; puncturing the venous system at a predetermined position; disposing the elongate instrument into the pericardial space at a predetermined location in the pericardial space; and implanting a pacemaker lead at the predetermined position.

### RELATED APPLICATIONS

The present application is related to U.S. Provisional Patent Applications Ser. No. 60/426,773, filed on Nov. 15, 2002; Ser. No. 60/476,487, filed on Jun. 6, 2003; 60/479,399, filed on Jun. 18, 2003; and Ser. No. 60/464,437, filed on Apr. 22, 2003, which are each incorporated herein by reference and to which priority is claimed pursuant to 35 USC 119.

### BACKGROUND OF THE INVENTION

1. Field of the Invention

The invention relates to the field of cardiology and in particular to apparatus and methods for pacemaker implantations.

2. Description of the Prior Art

In many cases pacemaker introducers are precurved for steerability and use in the coronary sinus. Being precurved, such introducers cannot be rotated for the purpose of setting a screw-in anchor into myocardium, since their distal ends wobble uncontrollably while being rotated. It is nevertheless advantageous to anchor an introducer when similarly implanting or anchoring the much more flexible pacemaker lead. If the pacemaker lead is not supported by a fixed-in-place introducer, the pacemaker lead itself can either push back and displace the introducer with the result that the pacemaker lead is implanted in the wrong place, or the pacemaker lead simply “spaghettis” or unpredictably folds on itself rather than being controllably driven in or implanted into the myocardial position selected by the surgeon.

Pacing of the left ventricle 14 diagrammatically shown in FIG. 1 is conventionally performed by placement or implantation of a wire or lead 10 in the coronary venous system 12 as diagrammatically shown in the cutaway view of the coronary vein 16 in FIG. 2. However, not every location of the wall of the left ventricle 14, which needs to be paced, is accessible to a lead through the coronary venous system 12 as can be readily appreciated by the vascular anatomy depicted of the coronary venous system 12 and coronary arterial system 18 depicted in the simplified perspective view of FIG. 2.

### BRIEF SUMMARY OF THE INVENTION

The invention is an introducer having a distal end comprising an anchor provided on the distal end of the introducer for attachment of the distal end of the introducer into tissue. The anchor attaches at or near the surface of a body cavity, for example in the vascular space, or more particularly in the cardial space. When the introducer is anchored in the cardial space a pacemaker lead is guided through the introducer and anchored therein while the introducer is anchored within cardial space.

The invention is directed to anchoring introducers and leads throughout the vascular space, including anchoring into any one of the walls of the heart chambers, into any vascular location, and into body cavities, usually closely related to the vascular system such as the pericardial space. The anchoring can be realized through a plurality of different means such as screw anchors, barbed anchors, piercing tools with barbs or distal inflatable balloons, grabbing tools or suction anchors.

In one embodiment the invention is defined as a method of implanting a pacemaker lead into the pericardial space or microvasculature of a heart comprising the steps of: disposing an elongate instrument into the venous system of the heart; exiting the venous system; disposing the elongate instrument or a different elongate instrument into the pericardial space or microvasculature at a predetermined location; and implanting a pacemaker lead at the predetermined position.

The step of implanting a pacemaker lead at the predetermined position comprises the step of implanting the lead in a position adjacent to, on or in the surface of the left ventricle.

Preferably, the step of implanting a pacemaker lead at the predetermined position comprises the step of implanting the lead in a position of optimized pacing efficacy.

In one embodiment the step of disposing an elongate instrument or a different elongate instrument into the pericardial space or microvasculature at a predetermined location comprises the step of disposing the elongate instrument or a different elongate instrument through the microvasculature into the pericardial space.

In another embodiment the step of disposing an elongate instrument or a different elongate instrument into the pericardial space or microvasculature comprises the step of disposing the elongate instrument or a different elongate instrument into a first venous bed.

In another embodiment the step of disposing an elongate instrument or a different elongate instrument into the pericardial space or microvasculature comprises the step of disposing the elongate instrument or a different elongate instrument into the vascular mesh.

In still another embodiment the step of disposing an elongate instrument or a different elongate instrument into the pericardial space or microvasculature comprises the step disposing the elongate instrument or a different elongate instrument from the first venous bed, through the vascular mesh into a second venous bed.

The method further comprises the step of anchoring the implanted pacemaker lead in the pericardial space or microvasculature.

In one embodiment the step of implanting the pacemaker lead comprises implanting the pacemaker lead in the microvasculature and further comprises the step of dilating the microvasculature prior to implanting the pacemaker lead therein.

In another embodiment the step of exiting the venous system comprises the step of puncturing a vein in the venous system.

In still another embodiment the step of exiting the venous system comprises the step of entering the vasculature communicated with the venous system.

In yet another embodiment the step of exiting the venous system comprises the step of exiting the vasculature and entering the pericardial space.

The step of disposing the elongate instrument or a different elongate instrument into the pericardial space or microvasculature at a predetermined location comprises the step of disposing an introducer, catheter, guidewire, balloon, dilator, needle and/or lead.

The invention is also defined as apparatus or a surgical kit of instruments for performing each of the foregoing steps separately or in any combination.

The invention is still further defined as an apparatus for implanting a pacemaker lead into heart tissue comprising an inner introducer which is steered into the heart; a first anchor provided on a distal end of the introducer; a pacemaker lead telescopically disposed through the inner introducer; and a second anchor provided on a distal end of the pacemaker lead.

The first anchor has an inner diameter large enough to permit telescopic disposition therethrough of the second anchor and pacemaker lead.

The apparatus may in some embodiments further comprise an outer biased introducer through which the inner introducer is telescopically disposed and steered to an implantation site.

In one embodiment the pacemaker lead is rotatable within the inner introducer and where the second anchor screws into the heart tissue at an implantation site and wherein the first anchor maintains the inner introducer in position while the second anchor screws into the heart tissue at the implantation site. The first anchor can be disengaged from the heart tissue at the implantation site, after the second anchor is implanted without dislodgement of the second anchor and pacemaker lead.

In a first embodiment the first anchor is a screw anchor with a first sense of screw advancement and where the second anchor is a screw anchor with a second sense of screw advancement opposite to the first sense of screw advancement.

In all embodiments it is possible that the outer introducer and inner introducer are separable, including sliceable, splittable, peelable, or tearable.

In yet another embodiment the second anchor is rotatable on and captured by the inner introducer and drivable by an elongate instrument. In this case the apparatus further comprises the elongate instrument and a lumen defined through the inner introducer through which the elongate instrument is disposed. The second anchor is typically, but not necessarily telescopically disposable from the distal end of the inner introducer.

The apparatus may further comprise a plurality of first anchors coupled to the inner introducer.

The invention contemplates that the inner, or outer both introducers may biased.

In an illustrated embodiment the first anchor comprising a fish-hook anchor.

The first anchor may be movably coupled to the inner introducer and is deployed when the inner introducer is telescopically advanced out of the distal end of the outer introducer. The first anchor is resiliently dispose within the inner introducer and automatically resiliently deployed when the inner introducer is telescopically advanced out of the distal end of the outer introducer. In one implementation the apparatus further comprises a wire coupled to the first anchor and which is operative when manipulated to rotate the anchor to extend out of or be retracted in the inner introducer. The first anchor is resiliently biased to be normally retracted within the inner introducer and where the wire is operated by applying a tensile force to rotate the first anchor to an extended configuration out of the inner introducer.

In still a further embodiment the apparatus further comprises an elongate instrument and a lumen defined through the inner introducer through which the elongate instrument is disposed, the first anchor being a piercing tool coupled to a distal end of the elongate instrument and having at least one barb disposed thereon. The first anchor comprises a plurality of barbs on the piercing tool, which may take the form of a plurality of stiff angled fibers disposed on the piercing tool.

In another embodiment the first anchor comprises a hollow needle with an inflatable tip balloon.

In still another embodiment the first anchor comprises a bimetallic wire which can be differentially tensioned and curved to form a temporary distal hook.

In all of the embodiments the apparatus may further comprise a hemostatic valve coupled to the inner introducer.

In yet more embodiments the first anchor is a suction device. In one example, the suction device comprises a suction cavity defined in the inner introducer with a peripheral lip to assist in allowing a suction attachment to the heart tissue. The suction cavity may be positioned on a lateral surface of the inner introducer.

The inner introducer has a lumen and the suction device comprises a means for providing suction to the lumen and communicating the suction to the distal orifice of the lumen at the distal tip of the inner introducer. The means for communicating the suction to the distal orifice of the lumen at the distal tip of the inner introducer comprises a central lumen defined through the inner introducer through which central lumen the pacemaker lead is disposed. In another embodiment the lumen is an auxiliary lumen defined through the inner introducer and where the means for communicating the suction to the distal orifice of the lumen at the distal tip of the inner introducer comprises a communication of the lumen with the distal orifice of the lumen at the distal tip of the inner introducer.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side cross sectional view of the distal end of an outer introducer comprised of a conventional precurved or biased outer introducer with an unbiased or flexible, but rotationally stiff inner telescopic introducer.

FIG. 2 is a side cross sectional view of the distal end of a second embodiment of the telescopic introducer, which is comprised of a biased introducer through which the pacemaker lead with the distal anchor is implanted.

FIG. 3 is a side cross sectional view of the distal end of a third embodiment of telescopic introducer which is comprised of a prebiased introducer through which the pacemaker lead with the distal anchor is implanted.

FIG. 4 is directed to another embodiment where an outer biased introducer is used to steer to the approximate site in the heart.

FIG. 5 is a simplified diagrammatic side cross section view of a human heart illustrating the pericardial space.

FIG. 6 is a simplified diagrammatic, partially cut-away side view of a human heart illustrating the vascular system of the heart.

FIG. 7 is a simplified diagram of the venous vascular mesh on the ventricular surface.

FIG. 8 is a simplified diagrammatic side cross section view of in enlarged scale of the region in circular dotted outline in FIG. 3 illustrating dilation of the microvasculature with a balloon.

FIG. 9 is a simplified diagrammatic side cross section view of FIG. 4 showing the disposition of a catheter into the microvasculature.

FIG. 10 is a simplified diagrammatic side cross section view of FIG. 5 showing the disposition of a guidewire and introducer into the microvasculature.

FIG. 11 is a simplified diagrammatic side cross section view of FIG. 5 showing the disposition of a pacemaker lead into the microvasculature.

FIG. 12 is a simplified diagrammatic side view of the disposition of an elongate instrument from a first venous bed through the venous vascular mesh and implantation in a second venous drainage area.

FIG. 13 is a simplified side cross-sectional view of a heart having a chamber into which an introducer with a suction anchor has been endovascularly disposed.

FIG. 14 is an enlarged simplified side cross-sectional view of the distal tip of the introducer of FIG. 13.

FIG. 15 is a side elevational view of the distal tip of the introducer of FIG. 14.

FIG. 16 is a diagrammatic side cross-sectional view of another embodiment of a suction anchor where the suction is provided to the distal orifice of catheter either through main lumen between the clearance of lumen and pacemaker lead or through an auxiliary lumen defined in the catheter with a distal communication with lumen at or near orifice.

FIG. 17 is a diagrammatic side cross-sectional view of an embodiment wherein suction is provided only through the axial lumen.

FIG. 18 is a diagrammatic side cross-sectional view of an embodiment wherein a grabbing tool is provided as the anchor through the axial lumen.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

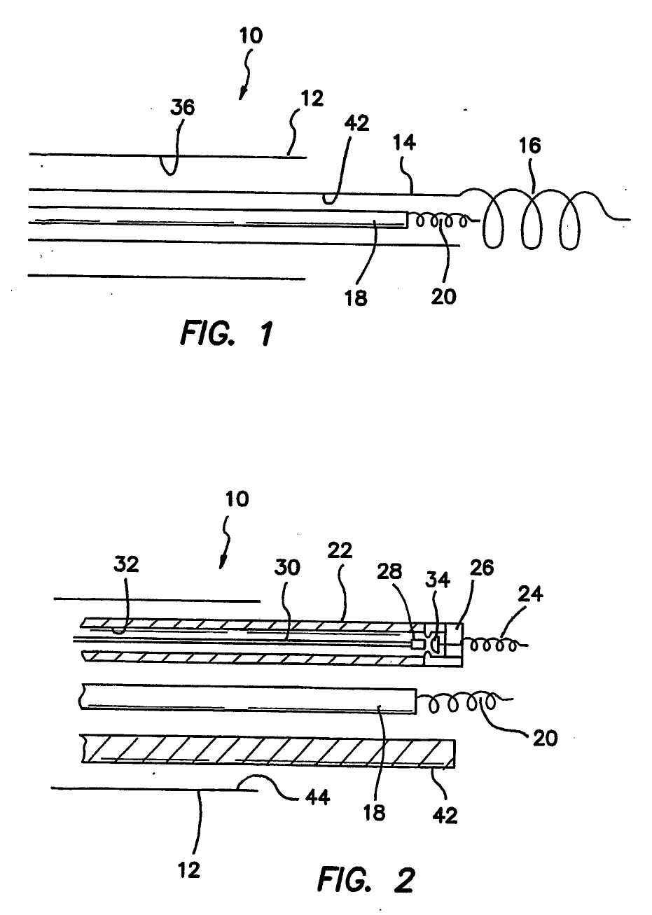

FIG. 1 is a side cross sectional view of the distal end of an outer introducer 10 comprised of a conventional precurved or biased outer introducer 12 with an unbiased or flexible, but rotationally stiff inner telescopic introducer 14. Alternatively, inner introducer 14 may be biased. Telescopic inner introducer 14 is provided at or near its distal end with a conventional screw-in anchor 16. Anchor 16 has an inner diameter which is large enough so that when pacemaker lead 18 is telescopically disposed through a axial lumen 42 in inner introducer 14 with its corresponding distal anchor 20, distal anchor 20 can be telescopically disposed through anchor 16 during fixation into the myocardium without engaging anchor 16.

Being in the preferred embodiment unbiased, inner introducer 14 easily stays on-position at the location defined by the distal end of outer introducer 12 when inner telescopic introducer 14 is rotated. When pacemaker lead 18 is then rotated and anchored, the support of anchored introducer 14 keeps pacemaker lead 18 supported and similarly on-position at the location defined by the distal end of outer introducer 12.

Once pacemaker lead 18 is implanted, inner introducer 14 can then be unscrewed without dislodging anchor 20 and removed. To enhance the compatibility of adjacent implantations of anchor 20 coaxially inside anchor 16, anchor 20 and anchor 16 can be provided with helicity of opposite senses. For example, if anchor 20 is a right-hand screw, anchor 16 is provided as a left-hand screw. In this manner, when anchor 16 is unscrewed, it is rotated clockwise when viewed from the proximal end of introducer 10. Any clockwise rotation transmitted by anchor 16 in any way to anchor 20 thereby serves to screw in or tighten anchor 20.

It must be understood that introducers 12 and 14 may be sliceable, tearable, peelable, or separable in any way now known or later devised, so that they are easily removed over any pacemaker hub or connector (not shown) at the proximal end of pacemaker lead 18.

FIG. 2 is a side cross sectional view of the distal end of a second embodiment of telescopic introducer 10 which is comprised of a biased introducer 22 through which pacemaker lead 18 with distal anchor 20 is implanted. A parallel lumen 32 is defined in the wall of introducer 22 through which a torsional wire 30 is disposed. Wire 30 has a termination 28 which is or can be coupled with a captive screw 34 which has a distal anchor 24. It is contemplated that screw 34 will be retained within lumen 32 until deployment, at which time it is then distally extended by being pushed by wire 30 and then rotated by wire 30 to fix anchor 24 into the adjacent myocardium. In this embodiment, anchors 20 and 24 are not telescopic, but are deployed in parallel.

It is to be understood that instead of a captive screw 34, wire 30 and anchor 24 may be integral and simply delivered through auxiliary lumen 32. A plurality of such auxiliary lumens 32 and wire 30/anchor 24 combinations may be provided and employed in a radial pattern at the distal end of introducer 22.

FIG. 2 is described above as a nontelescopic system. However, it must be understood as shown in FIG. 2 that introducer 22 may be biased or unbiased and similarly telescopically disposed through an axial lumen 44 of a biased outer introducer 12. As in the case of FIG. 1, the outer introducer 12 is used to steer inner introducer 22 to the approximately vicinity of the implantation site, inner introducer 22 is telescopically advanced out of introducer 12 and anchored in place. Pacemaker lead 18 is then telescopically advanced in lumen 42 and implanted using anchor 20.

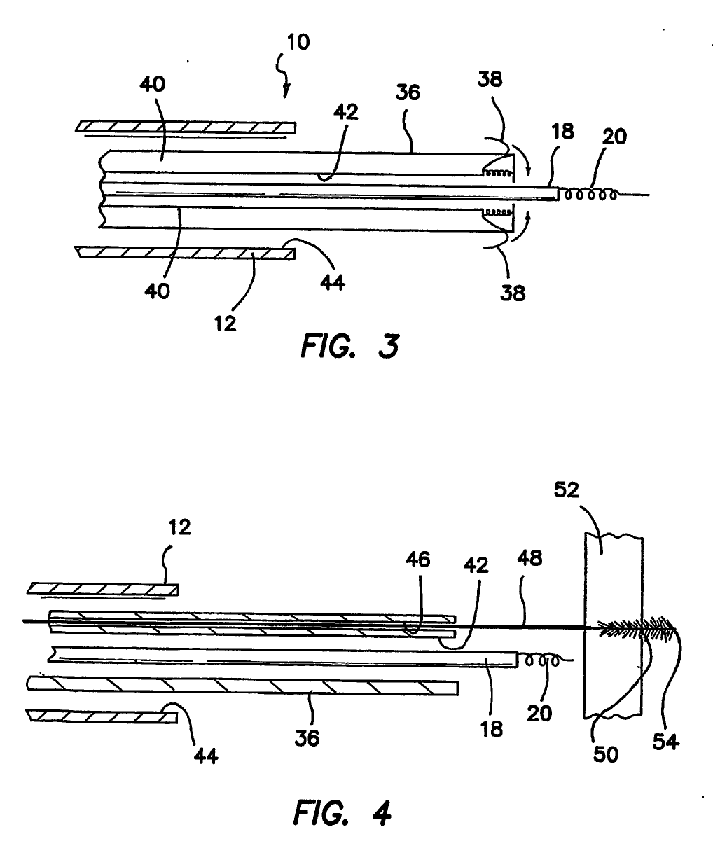

FIG. 3 is a side cross sectional view of the distal end of a third embodiment of telescopic introducer 10 which is comprised of a prebiased introducer 36 through which pacemaker lead 18 with distal anchor 20 is implanted. The distal portion or end of introducer 36 is provided with one or more barbless hooks or “fish hooks”, which are normally resiliently retained within recesses defined in the wall of introducer 36. Hooks 38 can be deployed by pulling tension wires 40 coupled to hooks 38 to rotate hooks 38 out of the recesses in introducer 36 to a position where they may penetrate radially adjacent myocardium or vascular tissue. Once hooks 38 are deployed by pulling on wires 40, pacemaker 18 is anchored from the end of anchored introducer 36. Once pacemaker lead 18 is anchored, the tension on wires 40 is released, allowing springs or other resilient means attached to hooks 38 to return them to their undeployed configuration with recesses within introducer 36.

Again it must be understood that while the embodiment of FIG. 3 is described above as a nontelescopic system, in another embodiment introducer 36 may be a prebiased on unbiased telescopic introducer disposed through axial lumen 44 in a biased outer introducer 12. As in the case of FIG. 1, the outer introducer 12 is used to steer inner introducer 36 to the approximately vicinity of the implantation site, inner introducer 36 is telescopically advanced out of introducer 12 and anchored in place. Pacemaker lead 18 is then telescopically advanced in lumen 42 and implanted using anchor 20. Thus, the parallel-lumen screw anchors of and hooks as shown in FIGS. 2 and 3 respectively can be substituted for the distal screw anchor 16 and single lumen introducer 14 of FIG. 1.

FIG. 4 is directed to another embodiment where outer biased introducer 12 is used to steer to the approximate site in the heart. Inner biased or unbiased introducer 36 is then telescopically disposed through axial lumen 44 in introducer 12 and position near or adjacent to a position on the heart wall 52 where pacemaker implantation is to occur. In the illustrated embodiment implantation is made into the intra-atrial septum 52 from the right atrium. A wire 48 is disposed in a parallel or auxiliary lumen 46 in inner introducer 36 and extends distally from the end of introducer 36. The distal end of wire 48 is or is fitted with a solid needle 54 which is able to puncture and to be forced through the smooth wall of intra-atrial septum 52. Wire 48 is further arranged or fitted with one or more flexible barbs, such as a plurality of flexible, but stiff fibers or filaments 50, which are biased to angle proximally on wire 48 like a brush. Needle 54 and filaments 50 can thus easily be advanced distally and pushed through intra-atrial septum 52 to serve as a temporary anchor of inner introducer 36 to intra-atrial septum, 52. Needle 54 may penetrate entirely through intra-atrial septum 52 into the left atrium. Wire 48 resists backing out or being pulled out of septum 52 by means of the proximally directed bias of filaments 50 which act as barbs. However, the anchoring strength is not so great that wire 48 cannot be later manually withdrawn taking needle 54 and filaments 50 with it.

With inner introducer 36 temporarily anchored to intra-atrial septum 52, pacemaker lead 18 with its distal screw anchor 20 is distally extended from axial lumen 42 in introducer 36 and screwed into intra-atrial septum 52. The anchored inner introducer 36 provides enough purchase or support to allow anchor 20 to be screwed into intra-atrial septum 52 without pacemaker lead 18 backing off or simply bending and collapsing against septum 52. The stiffness, degree of bias and number of filaments 50 are chosen to provide enough anchoring force that inner introducer 36, which is frictionally coupled to wire 48 in lumen 46, cannot be backed out by the reaction force applied to pacemaker lead 18, which is frictionally coupled to inner introducer 36 in lumen 42, by the screwing action of anchor 20 of pacemaker lead 18 into septum 52. Once pacemaker lead 18 is firmly anchored into or through septum 52, wire 48, filaments 50 and needle 54 can be pull out by applying sufficient tension to wire 48 from its proximal end.

It must be understood that while the temporary anchoring of wire 48 is shown by means of a plurality of filaments 50, there are many other equivalent ways by which temporary anchoring of wire 48 can be achieved. For example, needle 54 may be hollow and carry a small inflatable and deflatable tip balloon, or wire 48 and/or needle 54 may be a bimetallic wire which can be differentially tensioned and curved to form a temporary distal hook by means of an electrical current and ohmic heating of the bimetallic wire, or simply by exposure to the body heat.

FIG. 18 is another embodiment similar to FIG. 4 in which instead of the elongate tool or wire 48 in lumen 46 a stylet or other tool 56 is used which is terminated with a grabbing tool 58. In the illustrated embodiment grabbing tool 58 comprises a set of pincers with one or two movable jaws which are manipulated by stylet 56 to open and close, as well as to be telescopically disposable in lumen 46. The mechanism used to manipulate tool 58 may be any type of actuation device now known or later devised for actuating one or more opposing jaws, such as a hollow wire with an inner telescoping solid core wire with one jaw fixed to the hollow wire and the second jaw rotated about the first jaw and coupled to the core wire to rotate the second jaw about its pivot point on the first jaw. Alternatively, grabbing tool 58 may have one jaw fixed to the distal end of introducer 36 with the second jaw rotatable about a pivot point also fixed to the distal end of introducer 36 or the first jaw. The two jaws may be resiliently biased to a closed position. Style 56 then takes the form of a tension wire 56 to rotate the second jaw relative to the first jaw to open and close the grabbing tool 58. In any embodiment grabbing tool 58 is operative to temporarily pinch, seize, or grab hold of some heart or vascular tissue to temporarily fix introducer 36 in place. In the case where stylet 56 is telescopically slidable within lumen 46, style 56 can be vascularly disposed and grabbing tool 58 actuated to fix it to a location. Introducer 36 is then guided into position over anchored stylet 56 until the distal end of introducer 36 is proximate to or adjacent the tissue site to which the lead 18 is to be implanted. Stylet 56 may then be longitudinally fixed to introducer 36, such as by a proximal locking or pinching device (not shown), and then lead 18 implanted in a conventional manner with introducer 36 securely but temporarily fixed into position.

While this disclosure is directed to the anchoring of a catheter or introducer, it must be expressly understood that the disclosed catheter or introducer includes within its scope any sliceable, splittable, peelable, tearable or separable catheter or introducer now known or later devised, as well as catheters or introducers, which cannot be separated in any of these manners. In addition, whether or not a hemostatic valve is associated with the catheter or introducer of the invention, and if so, whether or not the hemostatic valve is separable or not, together with or separately from the catheter or introducer, is all expressly included within the scope of the disclosed invention.

For example, splittable valves of the type disclosed in Lee, “Splittable Hemostatic Valve and Sheath and the Method for Using the Same”, U.S. Pat. No. 5,125,904 (1992) and U.S. Pat. No. 5,312,355 (1994), which are incorporated herein by reference, are included. Catheters and introducers are included of the type as disclosed in: Kurth, “Permanent Catheter with an Exterior Balloon Valve and Method of Using the Same,” U.S. Pat. No. 5,792,118 (1998), “Method and Apparatus for Insertion of Elongate Instruments Within a Body Cavity,” U.S. patent application Ser. No. 09/708,150 (2000), “A Temporarily Secured Guidewire and Catheter for Use in the Coronary Venous System and Method of Using the Same,” U.S. patent application Ser. No. 10/365,890 (2003), “A Method and Apparatus for a Suction-Anchored Introducer for Pacemaker Implantation,” U.S. Provisional Patent Application Ser. No. 60/464,437 (2003), “Method and Apparatus for Implantation of Left Ventricular Pacing Leads Between the Epicardium and Pericardium,” U.S. Provisional Patent Application Ser. No. 60/426,773 (2002), and “A Tool for Placement of Dual Angioplasty Wires in the Coronary Sinus Vasculature,” U.S. Provisional Patent Application Ser. No. 60/408,385 (2002); Worley et. al., “Introducer for Accessing the Coronary Sinus of a Heart,” U.S. patent application Ser. No. 10/139,551 (2002), “A Telescopic, Peel-Away Introducer and Method of Using the Same,” U.S. patent application Ser. No. 10/139,554 (2002), “A Telescopic, Peel-Away Introducer and Method of Using the Same,” U.S. patent application Ser. No. 10/139,554 (2002), “A Telescopic Introducer with a Compound Curvature for Inducing Alignment and Method of Using the Same”, U.S. patent application Ser. No. 10/202,158; and Kurth et. al; “Introducer and Hemostatic Valve Combination and Method of Using the Same, “U.S. patent application Ser. No. 10/234,686 (2002), “A Compression Fitting for an Introducer Coupled to a Hemostatic Valve,” U.S. patent application Ser. No. 10/277,476 (2002), which are all incorporated herein by reference.

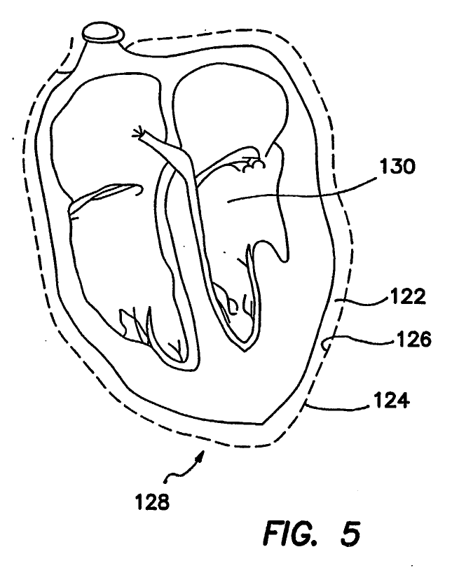

Consider now pacemaker anchoring in the pericardial space. As shown in the diagrammatic view of FIG. 5 the human heart 128 is contained within a conical sac of serous membrane called the pericardial sac or simply the pericardium 124 that encloses the heart and the roots of the great blood vessels of humans and vertebrates in general. The epicardium 126 is the visceral part of the pericardium 124 that closely envelops the heart. In between the epicardium 126 and the outer surface of the cardiac muscle and vasculature is a space lying above the coronary vasculature 112, 118 called the pericardial space 122. This pericardial space 122 may be void or partially filled with a lubricious fluid. In diseased states the pericardium may contain a substantial amount of fluid.

A pacemaker lead is implanted in a heart into the pericardial space, on or in the epicardium or in the microvasculature by: disposing an elongate instrument into the venous system of the heart; puncturing system at a predetermined position or entering the microvasculature of the venous system; disposing the elongate instrument into the pericardial space, epicardium or in the microvasculature at a predetermined location in the pericardial space, epicardium or in the microvasculature; and implanting a pacemaker lead at the predetermined position. It should be clear that the lead can be implanted either into the pericardial space or into the vascular mesh in or on the heart wall surface just adjacent to the pericardial space.

The step of implanting a pacemaker lead at the predetermined position comprises implanting the lead in a position on the surface of the left ventricle in a position of optimized pacing efficacy through the venous microvasculature on the ventricular surface or in the pericardial space.

In one embodiment the elongate instrument may be disposed into a first venous bed through the vascular mesh and subsequently into a second venous drainage bed for optimal positioning at or near the ventricular surface or adjacent pericardial space.

In either case the implanted pacemaker lead is then anchored in the venous microvasculature on the ventricular surface or in the pericardial space.

The microvasculature may also be dilated prior to implanting the pacemaker lead in order to allow for access of the guiding instrument or lead.

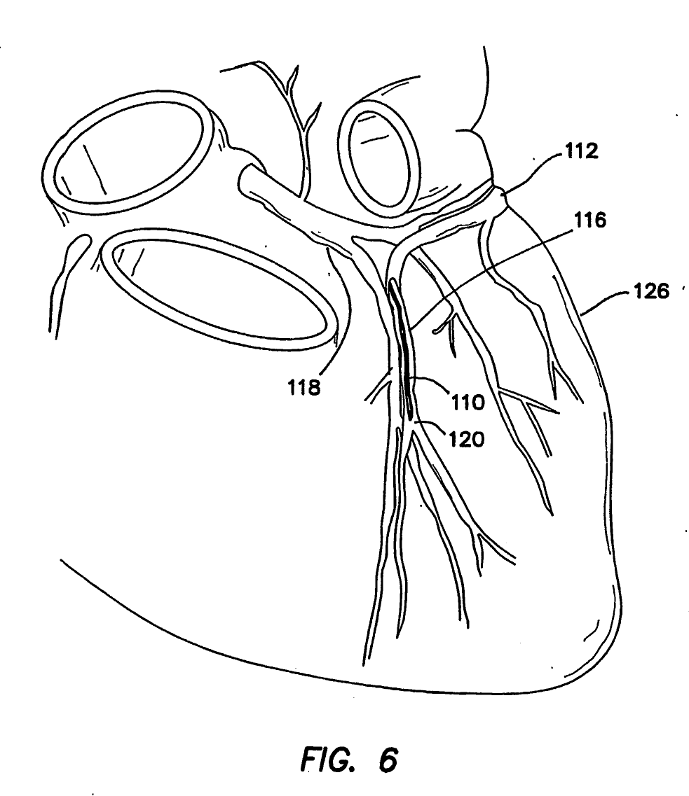

Consider first implantation of a lead into the pericardial space 122. In this embodiment the invention is directed to a method and apparatus in which a wire, catheter, lead, introducer or other instrument 110 is endovascularly disposed by conventional means into the coronary venous system 112 to a point 120 in the coronary venous system 112 where a puncture of the venous system 112 may take place as depicted in FIG. 6.

At this point 120, the coronary vein 116 is punctured or otherwise opened to allow the disposition of the wire, catheter, lead, introducer or other instrument 110 to be disposed through the vein 116 and then inserted, steered or disposed in the pericardial space 122 to the desired location on the heart's surface, or in this case in the vicinity of the left ventricle 130. Once in position it is anchored by conventional means in the pericardial space 122.

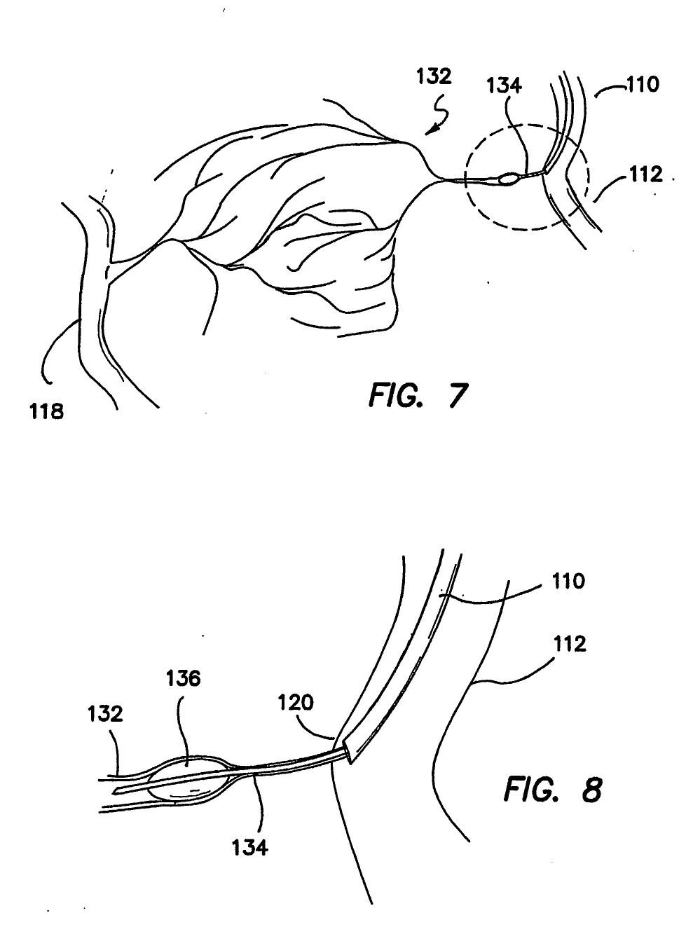

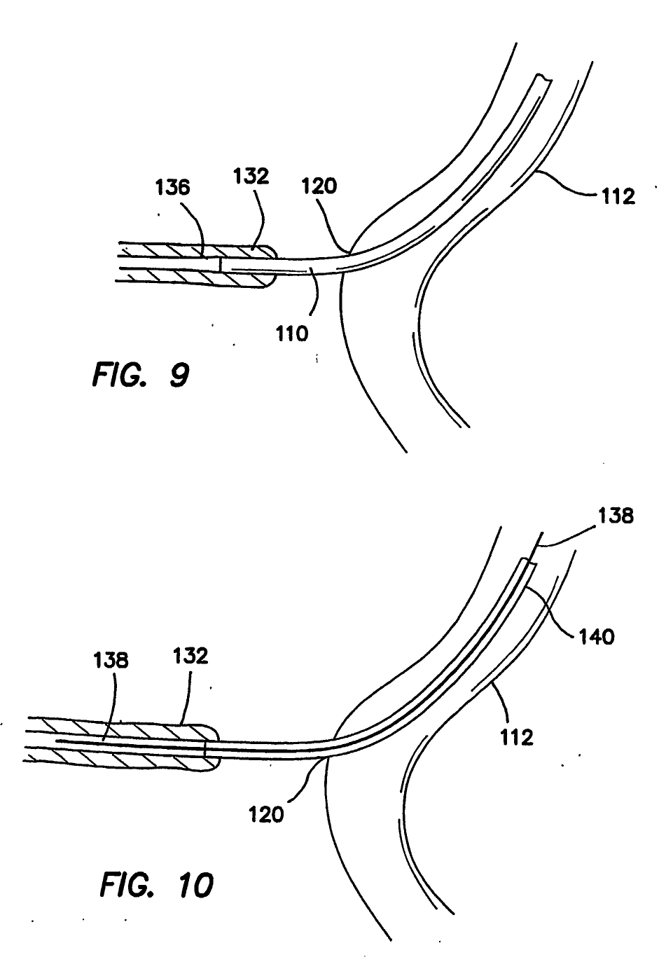

There are many means whereby the incision or puncture through the wall of vein 116 may be accomplished. A hollow or solid needle 134 shown in FIGS. 7 and 8 can be disposed through a catheter 110 and positioned at the venous site 120 selected. Advancement of the needle 134 beyond the distal tip of the catheter 110 allows the needle to puncture the vein 116 at the desired location 120. The vein 116 may also be punctured employing cutting or puncturing probes using ohmic heating, laser light, radiofrequency or microwave heating, ultrasonic or other energy sources, a balloon or blunt probe may also be used to open the vein into the pericardial space.

Once the vein 116 is punctured confirmation must be obtained that entry into the pericardial space 122 is accomplished. This can be practiced by injecting a contrast agent through the puncture site 120 into the pericardial space 122, obtaining an ultrasound image of the field of operation, or inserting a guidewire or other radio opaque means into the puncture site 120 for fluoroscopic confirmation.

With confirmation of entry into the pericardial space 122 a guidewire or probe 138 is then advanced into the space 122 through catheter 110, which may be removed and then followed, if desired, by an introducer or other introducing instrument 140 which is steerable or otherwise navigable to the desired location in the pericardial space 122 adjacent to or proximal to the desired location in the left ventricular wall as shown in FIG. 10.

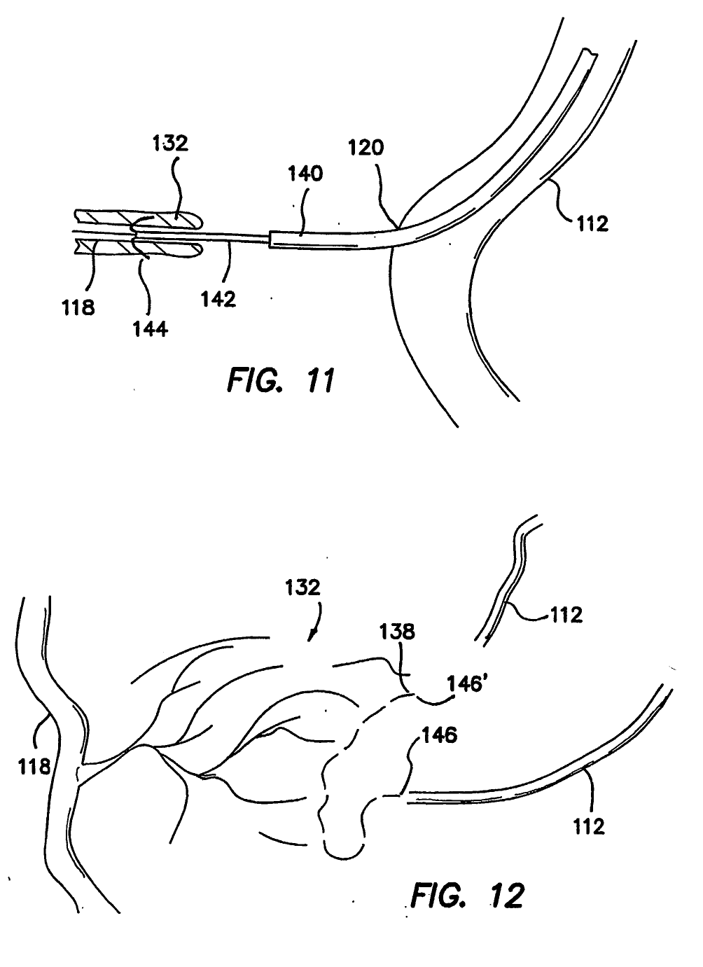

Finally, a pacing lead 142 is then brought or disposed at the desired location using the introducer or other introducing instrument 140 or the pacing lead 142 itself may be self-guiding as shown in FIG. 11. It is contemplated that once the desired location has been accessed; the pacing lead 142 will be anchored to the site in a conventional manner. Conventional anchoring means 144 can be employed or a mechanism with is optimized to the special environment of the pericardial space can be employed. In the case of patients who have cardiac bypass surgery, the pericardial space 22 often includes adhesive tissues, which provide a naturally adhesive or embedding tissue field and a pericardial-epicardial anchor 144 may not be required or is of less concern.

No restrictions or limitations are envisioned as being included which would in any way reduce the scope of the means whereby the wire, catheter, lead or other instrument 110, 138, 140, or 142 may be steered, by which the vein 116 is punctured, by which the vein is sealed around the wire, catheter, lead, other instrument, 110, 138, 140, or 142 or implanted pacing lead, 142 or by which the implanted pacing lead 142 is anchored at the desired location.

Consider now the implantation of a lead into the vascular mesh. Ventricular surface of the heart has disposed therein and/or thereon a microvasculature 132 as diagrammatically shown in FIG. 7 between the arterial system 118 and venous system 112 forming what comprises a vascular mesh. The vascular mesh is comprised of a multiplicity of small vessel radiating from the more distal portions of the coronary venous system. The vascular mesh subdivides into smaller and smaller multi-branched vessels and ultimately communicates to the capillary system in the heart walls. Blood drains from the heart muscle into the vascular mesh and then into the coronary veins. Generally, a flow path can be found through the vascular mesh communicating one vessel of the coronary venous system with another vessel of the coronary venous system.

The microvasculature 132 may also be opened or dilated with a balloon 136 or blunt instrument that opens the distal microvasculature 132 to allow for a catheter or other instrument 110 to be advanced. The balloon 136 may be withdrawn, or a central channel through a balloon catheter 110 may be used to withdraw needle 134, so that another catheter, lead or other instrument 110 can be deployed into the microvasculature 132.

In one embodiment access to the venous system 112 through the coronary sinus is accomplished using a fine, flexible 0.014 inch guidewire 138. The guidewire 138 is steered through a selected venous path to the very end of a venous bed 146 shown in FIG. 12. At the end of a venous bed 146, the vascular system 112, 118 communicates with an adjacent vascular bed through a vascular mesh 132 located on the epicardium 126 and also communicating with one or more other venous beds 146′. Theoretically, a path can be traced through the vascular mesh 32 between any two venous beds 146 and 146′ in the entire cardiac vascular system 112, 118. In theory the wire 138 can be advanced through the vascular mesh 132 into an adjacent or another venous bed 146 and ultimately looping back to the coronary sinus.

In this manner the wire 138 can be then steered from a first venous bed 146 to a selected position in a second venous bed 146′, which position 146 might be accessible or easily accessible through the coronary sinus and the second venous bed 146′, accessible as a practical matter only by a path through the coronary sinus 112, the first venous bed 146, the vascular mesh 132 and into the second venous bed 146′. Therefore, the ideal or, desired position for a pacing lead 142 becomes accessible even if located in the second venous bed 146′ through the first venous bed 146.

The pacemaker lead 142 is anchored in its position by virtue of its frictional engagement or intimacy with the terminal end of the first venous bed 146 and with the vascular mesh 132. If necessary, the end of the first venous bed 146 and the vascular mesh 132 can be opened by positioning an angioplasty balloon 136 on the guidewire 138 at the position of terminal constriction of the first and second venous beds 146, 146′ and in the vascular mesh 132. This allows for the easy passage then of a pacemaker lead 142 through the terminal constriction of the first and second venous beds 146, 146′ and the vascular mesh 132. In some cases an introducer 140 may be disposed through the terminal constriction of the first and second venous beds 146, 146′ and the vascular mesh 132 and employed to deliver the pacemaker lead 142. Removal of the introducer 140 leaves the lead 142 anchored in position in the second venous bed 146′ by virtue of its embedment in the terminal constriction of the first and second venous beds 146, 146′ and/or the vascular mesh 132.

Similarly, if the ideal or desired position for a pacing lead 142 happens not to lie in the vicinity of any venous bed, then direct access from the first venous bed 146 through the vascular mesh 32 can be achieved, using a pericardial-epicardial anchor 144 on the pacemaker lead tip. Use of the angioplasty balloon 136 as described above opens up access to the vascular mesh 132 and allows a steerable introducer 140 or lead 142 to then be selectively placed in the vascular mesh.

It is further possible that use of the balloon 136 may be used to intentionally rupture the microvasculature 132 allowing the lead 142 to then enter the pericardial space 122 and become anchored therein as described above in a manner similar to venous puncture.

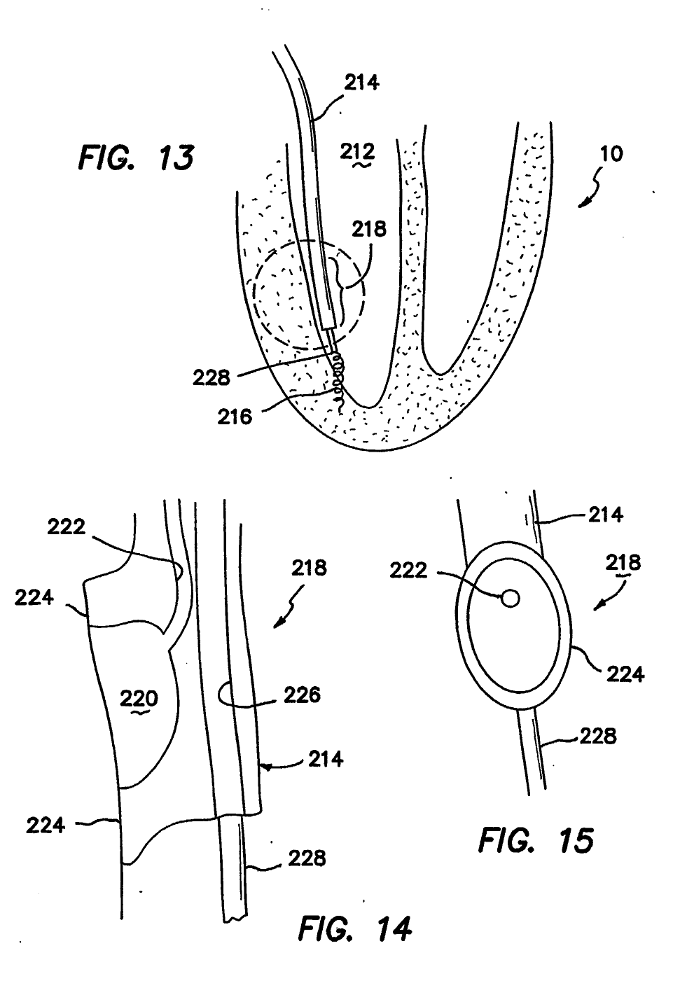

FIG. 13 is a simplified side cross-sectional view of a heart 210 having a chamber 212 into which an introducer 214 has been endovascularly disposed. Introducer 214 may be employed in an body cavity or portion of the vascular system. Introducer 214 has a suction anchor portion 218 on its distal end identified in the dotted portion of FIG. 13 and which is shown in enlarged simplified side cross-sectional view in FIG. 14.

Introducer 214 is provided with an axial lumen 226 through which a pacemaker lead 228 is or can be disposed. As shown in FIG. 13 pacemaker lead 228 has a tissue anchor or screw 216 on its distal end for attachment or embedding into the wall of heart 210. Any type of distal or other anchoring device now known or later devised may be used with pacemaker lead 228 without departing from the spirit and scope of the invention. In general, regardless whatever type of anchoring device 216 is used with pacemaker lead 228, it tends to push against pacemaker lead 228 and hence against introducer 210 and tends to dislodge or move introducer 210 away from its intended implantation position or configuration.

To provide for a noninvasive or nontraumatic anchoring of the distal end of introducer 210, the invention provides a suction anchor 218 for the distal end of introducer 210 as best shown in FIG. 14. Introducer 210 has its distal portion formed so that there is a suction cavity 220 defined therein and opening on one side of introducer 210. A lumen 222 communicates with cavity 220 and provides a means of providing and maintaining a suction on cavity 220. The periphery of cavity 220 is provided with a circumscribing lip 224 to facilitate sealing and suction attachment to the wall of heart 210, even when there are slight irregularities in the wall's surface as best seen in the side elevational view of FIG. 15. The amount of suction applied to cavity 220 is controlled proximally by the surgeon by means of a suction pump (not shown). Introducer 214 is therefore able to temporarily attach itself to a body cavity wall like a sucker fish without traumatic damage to the tissue where attachment is realized. Nevertheless, the degree of attachment is secure, but can be selectively and quickly released.

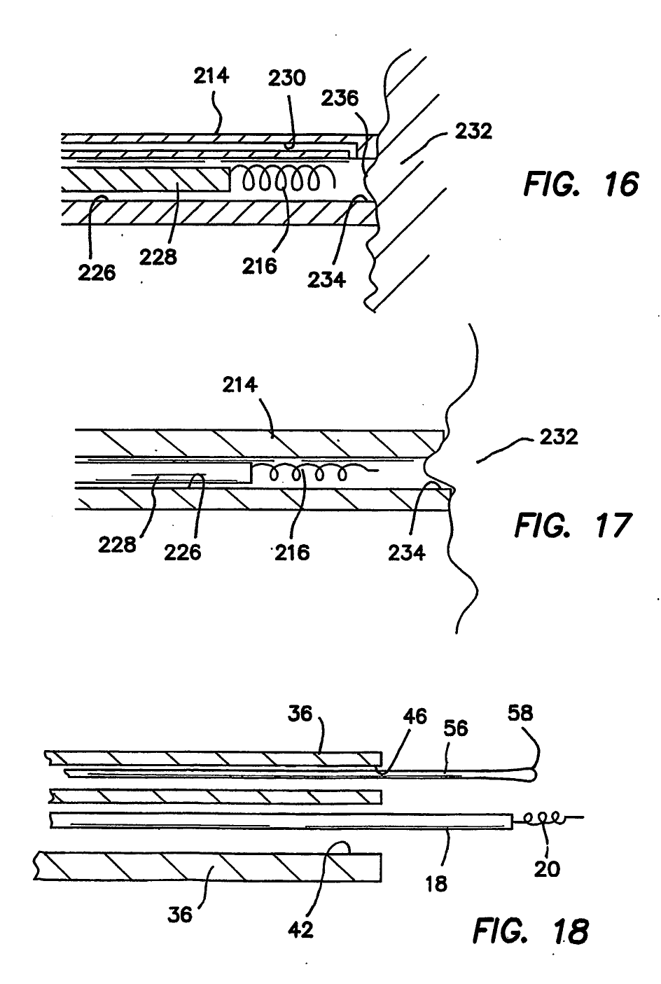

A preferred embodiment is shown in the diagrammatic side cross-sectional view of FIG. 16 wherein the suction is provided to the distal orifice 234 of catheter 214 either through main lumen 226 between the clearance of lumen 226 and pacemaker lead 218 or through an auxiliary lumen 230 defined in catheter 214 with a distal communication with lumen 226 at or near orifice 234. To clarify these alternative embodiments, FIG. 17 is a diagrammatic side cross-sectional view of an embodiment wherein suction is provided only through axial lumen 226. In either case the suction is provided at the distal orifice 234 of catheter 214 which is position into contact with tissue or heart wall 232. As seen in FIG. 16 the suction may draw some to the tissue 236 of wall 232 into orifice 234 and seal directly with wall 232. Anchor 216 may then be advanced in lumen 226 through orifice 234 and fixed in a conventional manner into tissue 236. Continuous suction is applied to lumen 226 during the fixation operation so that tissue 236 of wall 232 is keep in position in or adjacent to orifice 234. After conventional fixation of lead 228 is achieved. The suction is terminated, tissue 236 released, and catheter 214 is removed.

The embodiments of FIGS. 13-15 may require an additional cylindrical introducer to enable the device to be smoothly introduced through the tissue planes and inserted into the vascular system due to the irregularities in outline of the suction anchor on the distal tip of the catheter. While any structures departing from a smooth cylindrical envelope can be made of very soft or pliable materials, they may nonetheless present some resistance to easy introduction. On the other hand the embodiments of FIGS. 16 and 17 are preferred in that they are completely and smoothly cylindrical in their outer envelope and therefore are adaptable to over the wire techniques without the necessity for an additional introducer. The distal most end of catheter 214 may be modified to include forward extending lips or any other structure which would be beneficial in establishing a suction seal with the tissue.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. An introducer having a distal end comprising an anchor provided on the distal end of the introducer for attachment of the distal end of the introducer into tissue.

2. The introducer of claim 1 where the anchor attaches at or near the surface of a body cavity.

3. The introducer of claim 2 where the body cavity comprises a vascular space.

4. The introducer of claim 3 where the vascular space is a cardial space.

5. The introducer of claim 4 further comprising a pacemaker lead and where the pacemaker lead is introduced to the cardial space through the introducer and anchored therein while the introducer is anchored within cardial space.

6-29. (canceled)

30. An apparatus for implanting a pacemaker lead into heart tissue comprising:
an inner introducer which is steered into the heart;
a first anchor provided on a distal end of the introducer;
a pacemaker lead telescopically disposed through the inner introducer; and
a second anchor provided on a distal end of the pacemaker lead.

31. The apparatus of claim 30 where the first anchor has an inner diameter large enough to permit telescopic disposition therethrough of the second anchor and pacemaker lead.

32. The apparatus of claim 30 further comprising an outer biased introducer through which the inner introducer is telescopically disposed and steered to an implantation site.

33. The apparatus of claim 30 where the pacemaker lead is rotatable within the inner introducer and where the second anchor screws into the heart tissue at an implantation site and wherein the first anchor maintains the inner introducer in position while the second anchor screws into the heart tissue at the implantation site.

34. The apparatus of claim 33 where the first anchor can be disengaged from the heart tissue at the implantation site, after the second anchor is implanted without dislodgement of the second anchor and pacemaker lead.

35. The apparatus of claim 33 where the first anchor is a screw anchor with a first sense of screw advancement and where the second anchor is a screw anchor with a second sense of screw advancement opposite to the first sense of screw advancement.

36. The apparatus of claim 32 where the outer introducer and inner introducer are separable, including sliceable, splittable, peelable, or tearable.

37. The apparatus of claim 30 where the second anchor is rotatable on and captured by the inner introducer and drivable by an elongate instrument.

38. The apparatus of claim 37 further comprising the elongate instrument and a lumen defined through the inner introducer through which the elongate instrument is disposed.

39. The apparatus of claim 37 where the second anchor is telescopically disposable from the distal end of the inner introducer.

40. The apparatus of claim 30 further comprising a plurality of first anchors coupled to the inner introducer.

41. The apparatus of claim 30 where the inner introducer is biased.

42. The apparatus of claim 30 where the first anchor comprising a fish-hook anchor.

43. The apparatus of claim 33 where the first anchor is movably coupled to the inner introducer and is deployed when the inner introducer is telescopically advanced out of the distal end of the outer introducer.

44. The apparatus of claim 43 where the first anchor is resiliently dispose within the inner introducer and automatically resiliently deployed when the inner introducer is telescopically advanced out of the distal end of the outer introducer.

45. The apparatus of claim 43 further comprising a wire coupled to the first anchor and which is operative when manipulated to rotate the anchor to extend out of or be retracted in the inner introducer.

46. The apparatus of claim 45 where the first anchor is resiliently biased to be normally retracted within the inner introducer and where the wire is operated by applying a tensile force to rotate the first anchor to an extended configuration out of the inner introducer.

47. The apparatus of claim 30 further comprising an elongate instrument and a lumen defined through the inner introducer through which the elongate instrument is disposed, the first anchor is coupled to a distal end of the elongate instrument.

48. The apparatus of claim 47 where the elongate instrument comprises a stylet.

49. The apparatus of claim 47 where the first anchor comprises a grabbing tool.

50. The apparatus of claim 47 where the first anchor comprises a suction anchor.

51. The apparatus of claim 47 where the elongate instrument carries the first anchor on its distal end and is extendable from the distal end of the inner introducer.

52. The apparatus of claim 47 where first anchor is coupled to the inner introducer and where the elongate instrument is coupled to the first anchor to manipulate the first between at least and first and second configuration corresponding to anchoring or not anchoring to the heart tissue respectively.

53. The apparatus of claim 47 where the first anchor comprises a piercing tool coupled to a distal end of the elongate instrument where the piercing tool has at least one barb disposed thereon.

54. The apparatus of claim 53 where the first anchor comprises a plurality of barbs on the piercing tool.

55. The apparatus of claim 54 where the first anchor comprises a plurality of stiff angled fibers disposed on the piercing tool.

56. The apparatus of claim 30 where the first anchor comprises a hollow needle with an inflatable tip balloon.

57. The apparatus of claim 30 where the first anchor comprises a bimetallic wire which can be differentially tensioned and curved to form a temporary distal hook.

58. The apparatus of claim 30 further comprising a hemostatic valve coupled to the inner introducer.

59. The apparatus of claim 30 where the first anchor is a suction device.

60. The apparatus of claim 58 where the suction device comprises a suction cavity defined in the inner introducer with a peripheral lip to assist in allowing a suction attachment to the heart tissue.

61. The apparatus of claim 59 where suction cavity is on a lateral surface of the inner introducer.

62. The apparatus of claim 59 where inner introducer has a lumen and where the suction device comprises means for providing suction to the lumen and communicating the suction to the distal orifice of the lumen at the distal tip of the inner introducer.

63. The apparatus of claim 62 where means for communicating the suction to the distal orifice of the lumen at the distal tip of the inner introducer comprises a central lumen defined through the inner introducer through which central lumen the pacemaker lead is disposed.

64. The apparatus of claim 62 where the lumen is an auxiliary lumen defined through the inner introducer and where the means for communicating the suction to the distal orifice of the lumen at the distal tip of the inner introducer comprises a communication of the lumen with the distal orifice of the lumen at the distal tip of the inner introducer.
